(19) United States
(12) Patent Application Publication
(10) Pub. No.: US 2009/0324725 A1
(43) Pub. Date: Dec. 31, 2009

(54) PEG-MODIFIED HYDROXYAPATITE, PHARMACEUTICAL USING THE SAME AS BASE MATERIAL AND PRODUCTION PROCESS THEREOF

(75) Inventors: Kazunao MASUBUCHI, Yokohama-shi (JP); Junichi Minowa, Yokohama-shi (JP); Kazuo Watanabe, Tokyo (JP); Isao Umeda, Yokohama-shi (JP)

Correspondence Address: OBLON, SPIVAK, MCCLELLAND MAIER & NEUSTADT, L.L.P., 1940 DUKE STREET, ALEXANDRIA, VA, 22314, US

(73) Assignee: EBARA CORPORATION, Ohta-ku (JP)

(21) Appl. No.: 12/358,486

(22) Filed: Jan. 23, 2009

(30) Foreign Application Priority Data
Jan. 25, 2008 (JP) 2008-015444
Jun. 12, 2008 (JP) 2008-153764

(51) Int. Cl. A61K 9/14; A61K 38/00; A61K 31/7088; A61K 31/7028; A61K 31/496

(52) U.S. Cl. 424/489; 514/2; 514/44 A; 514/44 R; 514/29; 514/254.07

(57) ABSTRACT

The present invention provides a PEG-modified HAP having a high degree of safety and novel functions by modifying the surface of hydroxyapatite particles with a polyethylene glycol derivative, applications thereof, and a production process of the same. The PEG-modified HAP of the present invention is a substance in which hydroxyapatite having a particle diameter of 50 μm to 10 nm is bonded to a polyethylene glycol derivative having a carboxyl group as a terminal functional group through —O(CO) bonds, and the carbon content thereof is 10 to 0.1%. In addition, the present invention is a substance composed of this substance and a pharmaceutical active ingredient or pharmaceutical additive, in which the weight ratio of the pharmaceutical active ingredient is 1 to 30%, and the substance is obtained by treating hydroxyapatite having a particle diameter of 50 μm to 10 nm and an active ester of polyethylene glycol derivative having a carboxyl group as a terminal functional group in an anhydrous organic solvent.

### TECHNICAL FIELD

Microparticles for carrying drugs can be effectively used for a variety of drug forms: oral, intravenous, subcutaneous, transpulmonary or transnasal administrations by adjusting their size or by suitably modifying the microparticles. In addition, in terms of function, they can be effectively used to selectively deliver a drug to the liver, lungs or inflammatory site and the like, control drug release, mask unpleasant taste or improve intestinal absorption and the like.

Known examples of such microparticles include liposomes, polymer micelles, protospheres (registered trademark), resins and inorganic particles (inorganic microspheres or nanospheres) such as silica gel, zeolite or hydroxyapatite. The present invention relates to a novel polyethylene glycol-modified hydroxyapatite (abbreviated as PEG-modified HAP) in which the surface of hydroxyapatite is modified with polyethylene glycol (PEG), applications thereof and a production process of the same.

### BACKGROUND ART

Hydroxyapatite (hereinafter referred to as HAP) is a basic component of bones and teeth, has high bioaffinity and easily adsorbs sugars and proteins. Consequently, HAP is widely used as a pharmaceutical base material such as materials for repairing bones and teeth, column packing material, drug transporter or cell culture substrate. The surface of HAP is being required to be chemically modified with functional polymers and biologically active substances in order to more fully take advantage of its characteristics. However, since the hydroxyl groups on the HAP surface serving as the footholds for such modification have low reactivity, it is difficult to uniformly bond organic compounds such as functional polymers or biologically active substances.

Chemical modification of apatite particles is reported by Liu Qiug, et al. involving the bonding of polyethylene glycol to nanoapatite particles using hexamethylene diisocyanate (Biomaterials (1998), 19(11-12), 1067-1072). In addition, Furuzono, et al. succeeded in developing a transcutaneous device in which a silane coupling agent having amino groups is bonded to apatite particles followed by bonding with a silicone sheet by means of polyacrylic acid using a condensation reaction between carboxylic acid and amino groups (J. Biomed. Mater. Res. (2001), 56(1), 9-16). Moreover, Tanaka, et al. succeeded in introducing highly reactive organic functional groups onto the surface of porous HAP and then covalently bonding an organic substance to the surface of the porous HAP using a silane coupling agent having two or more types of functional groups and an isocyanate compound, and this is indicated as being able to be widely used in applications such as a chromatography column packing material, DDS carrier, ion exchange medium, cell culture substrate or implant (Japanese Unexamined Patent Application, First Publication No. 2003-342011).

However, since a bifunctional linker reagent is used in each of their production processes, crosslinking between hydroxyapatite particles can inevitably not be avoided, resulting in the problem of the crosslinked hydroxyapatite particles being present as by-products. In addition, since highly reactive silane coupling agents and isocyanate compounds are used, the safety of these residual reactive functional groups is also considered to be present problems.

[Patent Document 1] Japanese Unexamined Patent Application, First Publication No. 2003-342011

[Non-Patent Document 1] Biomaterials (1998), 19(11-12), 1067-1072

[Non-Patent Document 2] J. Biomed. Mater. Res. (2001), 56(1), 9-16

### DISCLOSURE OF THE INVENTION

### Problems to be Solved by the Invention

An object of the present invention is to provide a PEG-modified HAP having a high degree of safety and novel functions by modifying the surface of hydroxyapatite particles with a polyethylene glycol derivative without using a bifunctional linker, applications using the same, and a production process of the same.

### Means for Solving the Problems

As a result of conducting extensive studies to solve the aforementioned problems, the inventors of the present invention succeeded in introducing polyethylene glycol onto the surface of HAP with —O(CO) bonds using a polyethylene glycol derivative having a carboxyl group as a terminal functional group. Moreover, the inventors of the present invention found that a drug delivery system (DDS), in which various pharmaceuticals are loaded onto the novel PEG-modified HAP, can be effectively used for a variety of drug forms: oral, intravenous, subcutaneous, transpulmonary or transnasal administrations, and can be effectively applied to selectively deliver a drug to the liver, lungs or inflammatory site and the like, control drug release, mask unpleasant taste or improve intestinal absorption and the like.

This PEG-modified HAP can be expected to maintain the high mechanical strength and ability to adsorb various substances, which are characteristics of apatite, while also having properties such as retention in blood. Consequently, it can be widely used as a DDS carrier as well as chromatography column packing material, ion exchange medium, cell culture substrate or implant and the like.

Namely, the present invention provides that described in (1) to (23) below:

(1) a substance in the form of a mixture comprising hydroxyapatite having a particle diameter of 50 μm to 10 nm and a polyethylene glycol derivative having a carboxyl group as a terminal functional group, wherein the carbon content is 10 to 0.1%;
(2) a substance in which hydroxyapatite having a particle diameter of 50 μm to 10 nm is bonded to a polyethylene glycol derivative having a carboxyl group as a terminal functional group through —O(CO) bonds, wherein the carbon content is 10 to 0.1%;
(3) a substance comprising the substance described in (1) above and a pharmaceutical active ingredient, wherein the weight ratio of the pharmaceutical active ingredient is 1 to 30%, or a substance comprising the substance described in (1) above, a pharmaceutical active ingredient and a pharmaceutical additive, wherein the weight ratio of the pharmaceutical active ingredient is 1 to 30%;
(4) a substance comprising the substance described in (2) above and a pharmaceutical active ingredient, wherein the weight ratio of the pharmaceutical active ingredient is 1 to 30%, or a substance comprising the substance described in (2) above, a pharmaceutical active ingredient and a pharmaceutical additive, wherein the weight ratio of the pharmaceutical active ingredient is 1 to 30%;
(5) a pharmaceutical prepared from the substance described in (3) or (4) above;
(6) the substance described in (3) or (4) above, wherein the pharmaceutical active ingredient is an siRNA, aptamer, RNA, DNA, peptide or protein;
(7) the substance described in (3) or (4) above, wherein the pharmaceutical active ingredient is clarithromycin;
(8) the substance described in (3) or (4) above, wherein the pharmaceutical active ingredient is itraconazole;
(9) the substance described in (3) or (4) above, wherein the pharmaceutical active ingredient is siRNA;
(10) a method for obtaining the substance described in (1) or (2) above of the submicron size by treating submicron-sized hydroxyapatite and an active ester of a polyethylene glycol derivative having a carboxyl group as a terminal functional group in an anhydrous organic solvent;
(11) a method for obtaining the substance described in (3) or (4) above by treating a submicron-sized substance described in (1) or (2) above and a pharmaceutical active ingredient or pharmaceutical additive in an organic solvent;
(12) the substance described in (3) or (4) above, wherein the pharmaceutical active ingredient is candesartan;
(13) the substance described in (3) or (4) above, wherein the pharmaceutical active ingredient is candesartan cilexetil;
(14) the substance described in (3) or (4) above, wherein the pharmaceutical active ingredient is a double-stranded siRNA having the sequence of 5′-GUGAAGUCAACAUGCCUGCTT-3′ (SEQ ID NO. 1) and 5′-GCAGGCAUGUUGACUUCACTT-3′ (SEQ ID NO. 2);
(15) the substance described in (3) or (4) above, wherein the pharmaceutical active ingredient is a double-stranded siRNA having the sequence of 5′-CUUACGCUGAGUACUUCGATT-3′ (SEQ ID NO. 3) and 5′-UCGAAGUACUCAGCGUAAGTT-3′ (SEQ ID NO. 4);
(16) the substance described in (3) or (4) above, wherein the pharmaceutical active ingredient is etoposide;
(17) the substance described in (3) or (4) above, wherein the pharmaceutical active ingredient is nelfinavir mesylate;
(18) the substance described in (3) or (4) above, wherein the pharmaceutical active ingredient is simvastatin;
(19) the substance described in (3) or (4) above, wherein the pharmaceutical active ingredient is 7-ethyl-10-hydroxy-camptothecine;
(20) the substance described in (3) or (4) above, wherein the pharmaceutical active ingredient is paclitaxel;
(21) the substance described in (3) or (4) above, wherein the pharmaceutical active ingredient is saquinavir mesylate;
(22) the substance described in (3) or (4) above, wherein the pharmaceutical active ingredient is insulin; and,
(23) the substance described in (3) or (4) above, wherein the pharmaceutical active ingredient is bromocriptine mesylate.

### EFFECTS OF THE INVENTION

The following effects can be demonstrated by the present invention.

(1) Use of the PEG-modified HAP of the present invention enables even a poorly soluble pharmaceutical substance to be treated in the manner of a soluble substance, facilitating administration of a drug into the body and improving blood retention in the body.
(2) Modifying the surface of HAP with PEG makes it possible to prevent aggregation of HAP particles.
(3) Use of the PEG-modified HAP of the present invention in a base material makes it possible to prevent aggregation of particles even in the case of HAP particles loaded with an active ingredient.

### BRIEF DESCRIPTION OF THE DRAWINGS

FIG. 1 is a graph showing the particle size distribution of PEG-modified HAP of Example 1.

FIG. 2 is a graph showing the particle size distribution of a pharmaceutical composed of PEG-modified HAP and clarithromycin of Example 2.

FIG. 3 is a graph showing the particle size distribution of a pharmaceutical composed of PEG-modified HAP and itraconazole of Example 3.

FIG. 4A is a fluorescence micrograph (×1000) of PEG-modified HAP coated with fluorescein-labeled siRNA of Example 4, while FIG. 4B is a fluorescence micrograph (×1000) of PEG-modified HAP coated with rhodamine-labeled siRNA.

FIG. 5 is a graph showing the elution rate of candesartan over time (min) according to the results of Example 18.

FIG. 6 is a graph showing time-based concentration changes (hours) in plasma following intravenous injection to rats according to the results of Example 19.

FIG. 7 is a graph showing time-based concentration changes (hours) in plasma following oral administration to rats according to the results of Example 20.

FIG. 8 is a graph showing time-based concentration changes (hours) in plasma following intravenous injection to rats according to the results of Example 21.

FIG. 9 is a graph showing time-based concentration changes (hours) in plasma following oral administration to rats according to the results of Example 22.

FIG. 10 is a graph showing cytotoxicity against A549 cells according to the results of Example 23.

FIG. 11 is a fluorescence micrograph of cells 4 hours after a transfection test in A549 cells according to the results of Example 24.

FIG. 12 is a confocal laser micrograph of the results of Example 26.

FIG. 13 is a confocal laser micrograph of the results of Example 27

### BEST MODE FOR CARRYING OUT THE INVENTION

The following provides a detailed explanation of the present invention.

The present invention relates to a PEG-modified HAP having a high degree of safety and novel functions obtained by bonding a polyethylene glycol derivative to the surface of hydroxyapatite particles with a monofunctional polyethylene glycol derivative without using a bifunctional linker, applications thereof, and a production process of the same.

The HAP subjected to PEG modification may be an HAP solid having a large number of pores (air holes) or HAP not having very high porosity.

HAP is a compound having the general formula of Ca₅ (PO₄)₃OH, and includes a group of compounds referred to as calcium phosphates, such as CaHPO₄, Ca₃(PO₄)₂, Ca₄O(PO₄)₂, Ca₁₀(PO₄)₆(OH)₂, CaP₄O₁₁, Ca(PO₃)₂, Ca₂P₂O₇ or Ca(H₂PO₄)₂.H₂O according to the non-stoichiometric properties of reactions thereof. In addition, since HAP has as a fundamental component thereof a compound represented by the compositional formula Ca₅(PO₄)₃OH or Ca₁₀(PO₄)₆(OH)₂, a portion of the Ca component may be substituted with one or more types of substituents selected from the group consisting of Sr, Ba, Mg, Fe, Al, Y, La, Na, K, H and the like. In addition, a portion of the (PO₄) component may be substituted with one or more types of substituents selected from the group consisting of VO₄, BO₃, SO₄, CO₃, SiO₄ and the like. Moreover, a portion of the (OH) component may be substituted with one or more types of substituents selected from the group consisting of F, Cl, O, CO₃ and the like. In addition, a portion of each of these components may also be defective. Since a portion of the PO₄ and OH components of apatite of bone in the body are normally substituted with CO₃, entrance of CO₃ from the air and partial substitution into each component (on the order of 0 to 10% by weight) is permitted during production of the present composite biomaterial.

Furthermore, HAP may be adopt an ordinary microcrystalline, amorphous or crystalline form, as well as be in the form of an isomorphic solid solution, substituted solid solution or interstitial solid solution, and may contain non-quantum theory defects. In addition, the atomic ratio of calcium and phosphorous (Ca/P) in HAP is preferably within the range of 1.3 to 1.8 and more preferably within the range of 1.5 to 1.7. This is because, if the atomic ratio is within the range of 1.3 to 1.8, bioaffinity is enhanced since the composition and crystal structure of apatite (calcium phosphate compounds) in the product is able to adopt a composition and structure similar to apatite present in vertebrate bone.

Although the HAP subjected to PEG modification can be prepared using a known method, a commercially available product such as Hydroxyapatite nanopowder manufactured by Aldrich may also be used.

Although a commercially available high-purity, monofunctional activated PEG modifier is used for the “monofunctional polyethylene glycol derivative” used in the present invention, it is not limited thereto. Whether or not the PEG moiety is linear or branched, or the molecular weight of the PEG moiety and the like can be arbitrarily selected and adjusted according to the purpose.

Although an anhydrous organic solvent, and particularly dimethylformamide (DMF), dimethylsulfoxide (DMSO), acetic acid, acetone, tetrahydrofuran (THF), ethyl acetate or dichloromethane, can be used for the reaction solvent during modification, dimethylsulfoxide (DMSO) or acetone, which belong to class 2 to 3 as described in residual solvent guidelines for pharmaceuticals, is particularly preferable. The reaction temperature ranges from cooling with ice to 100° C., the reaction time is 2 to 72 hours, and the “monofunctional polyethylene glycol derivative” is used in large excess (1 to 0.1 g) per 1 g of HAP. Following completion of the reaction, residual excess “monofunctional polyethylene glycol derivative” and a by-product in the form of N-hydroxysuccinimide can be removed by washing with the organic solvent used in the reaction and filtering, and insoluble matter is vacuum dried to obtain PEG-modified HAP. Although the carbon content of the PEG-modified HAP can be adjusted to 0.1 to 10% by adjusting the amount of PEG modification reagent, it is preferably about 1 to 3% in particular.

The PEG-modified HAP of the present invention can be used as a DDS carrier by adsorbing a pharmaceutical active ingredient. Since both HAP and PEG have high biocompatibility, they can be used without reservation for delivering drugs into the body (J. Mater. Sci. (2000), 11(2), 67-72).

A drug can be delivered to a target organ more reliably by bonding a specific ligand to the target organ. In addition, in the case a pharmaceutical active ingredient is poorly soluble causing an injection preparation to be unable to or be poorly absorbed in the intestine, adsorbing a pharmaceutical active ingredient to submicron-sized PEG-modified HAP makes it possible to indirectly prepare pharmaceutically active ingredients at the submicron size, enabling them to be widely applied to the development of injection preparations and improvement of oral absorption. Moreover, substances in which pharmaceutical active ingredients in the form of RNA, DNA or a protein and the like are adsorbed to submicron-sized PEG-modified HAP can be applied as promising DDS for these pharmaceutical active ingredients.

A substance composed of PEG-modified HAP and a pharmaceutical active ingredient or pharmaceutical additive can be prepared using the following method. After dissolving the pharmaceutical active ingredient or pharmaceutical additive in a solvent belonging to class 2 to 3 described in residual solvent guidelines for pharmaceuticals, such as DMSO, ethanol (EtOH) or acetone, adding the PEG-modified HAP at a weight ratio of 90%, and subjecting to ultrasonic treatment at room temperature, the entire amount of the suspension is freeze-dried or removed of solvent by distilling under reduced pressure to obtain a substance as described in the claims. The loading ratio of the pharmaceutical active ingredient or pharmaceutical additive to the PEG-modified HAP can be adjusted to 1 to 30%, although dependent upon the pharmaceutical active ingredient or pharmaceutical additive, and is preferably about 10% in particular.

Although the following provides a more detailed explanation of the present invention through examples thereof, the present invention is not limited by these examples.

### Example 1

### Preparation of PEG-Modified HAP

### (1) Preparation of PEG-Modified HAP

200 mg of a polyethylene glycol (PEG) modifier (NOF, Sunbright ME-020CS) were added to a 20 ml of an acetone suspension containing 200 mg of Hydroxyapatite nanopowder (Aldrich, 677418) followed by radiating with ultrasonic waves (frequency: 28 kHz, output: 100 W) for 30 minutes. After stirring the suspension for 18 hours at room temperature, the suspension was separated by centrifugation (9000×g, 20° C., 30 minutes) followed by removing the supernatant by decanting. After washing the precipitate twice with acetone (20 ml×2), the precipitate was dried for 18 hours at 50° C. under reduced pressure to obtain 158 mg of PEG-modified HAP in the form of a white powder.

### (2) Measurement of Residual Solvent and PEG Modification Rate

The results of quantifying the residual solvent present in the prepared PEG-modified HAP by gas chromatography (GC) and quantifying the PEG modification rate in the form of the carbon content as determined by CHN reduction analysis are shown below.

Residual solvent (acetone) concentration: <100 μg/g

Carbon content: 2.01%<

<GC Analysis Conditions>

Apparatus: HP-589011 System (Hewlett Packard)

Column: DB-624, 75 mm×0.53 mm, membrane thickness: 0.3 μm

Column temperature: 40° C.→260° C.

Carrier gas: Helium, 7 psi

Detector: Hydrogen flame ionization detector (FID) 250° C.

<CHN Reduction Analysis Conditions>

Analyzer: Vario EL III (Elementar Analysensysteme GmbH)

Combustion oven temperature: 950° C.

Reduction oven temperature: 500° C.

Helium flow rate: 200 ml/min

Oxygen flow rate: 30 ml/min

Combustion time: 90 sec

### (3) Measurement of Particle Diameter Distribution

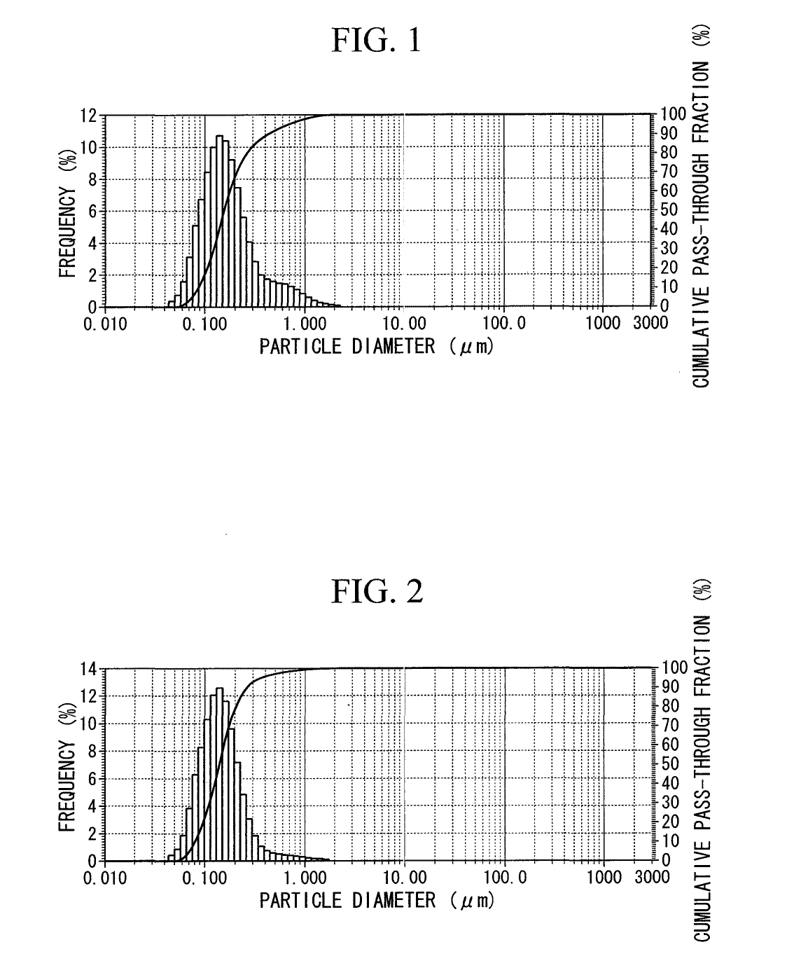

1 mg of the prepared PEG-modified HAP was suspended in Milli-Q water (15 ml) and irradiated for 5 minutes with ultrasonic waves (frequency: 28 kHz, output: 100 W) followed by measurement of particle diameter (measuring instrument: Horiba Laser Diffraction/Scattering Particle Diameter Distribution Measuring System LA-950) The results are shown in FIG. 1.

### Example 2

### Preparation of Substance Composed of PEG-Modified HAP and Clarithromycin

### (1) Preparation of Substance Composed of PEG-Modified HAP and Clarithromycin

A DMSO solution (2 ml) of clarithromycin (8 mg) was added to PEG-modified HAP (100 mg) followed by radiating for 2 minutes with ultrasonic waves (frequency: 28 kHz, output: 100 W). The suspension was freeze-dried to obtain 108.6 mg of a white powder. This was further dried for 36 hours at 50° C. under reduced pressure to obtain 108.1 mg of the target substance in the form of a white powder.

### (2) Measurement of Drug Adsorption Rate

1 ml of acetonitrile was added to 10 mg of the product followed by irradiating for 5 minutes with ultrasonic waves (frequency: 28 kHz, output: 100 W). The suspension was centrifuged (9000×g, 20° C., 3 minutes) and the supernatant was filtered with a 0.22 μm filter to obtain an HPLC sample. As a result of HPLC analysis, 0.74 mg of clarithromycin was confirmed to be contained in 10 mg of the product. Yield: 7.4% (w/w).

<HPLC Analysis Conditions>
Instrument: Waters Alliance 2695 Separations Module, Waters 2487 Dual λ Absorbance Detector
Column: Atlantis dC18, particle size: 3.0 μm, 3.9 mm×100 mm (Waters)
Mobile phase: A: 0.67 mol/L potassium dihydrogen phosphate reagent, B: acetonitrile, A:B=65:35 (v/v)
Flow rate: 1.0 ml/min
Detection wavelength: 210 nm
Retention time: 7.1 min

### (3) Measurement of Particle Diameter Distribution

1 mg of the product was suspended in Milli-Q water (15 ml) and irradiated for 5 minutes with ultrasonic waves (frequency: 28 kHz, output: 100 W) followed by measurement of particle diameter. The results are shown in FIG. 2.

### Example 3

### Preparation of Substance Composed of PEG-Modified HAP and Itraconazole

### (1) Preparation of Composition

A DMSO solution (4.8 ml) of itraconazole (24 mg) was added to PEG-modified HAP (300 mg) followed by irradiating for 2 minutes with ultrasonic waves (frequency: 28 kHz, output: 100 W). This suspension was freeze-dried to obtain 324.3 mg of a white powder. After suspending this in Milli-Q water (15 ml), an aqueous solution of sodium chondroitin sulfate (10 mg/ml) (0.3 ml) was added followed by irradiating for 2 minutes with ultrasonic waves (frequency: 28 kHz, output: 100 W). This suspension was then freeze-dried to obtain 322.6 mg of a white powder.

### (2) Measurement of Drug Adsorption Rate

1 ml of acetonitrile was added to 10 mg of the product followed by irradiating for 5 minutes with ultrasonic waves (frequency: 28 kHz, output: 100 W). The suspension was centrifuged (9000×g, 20° C., 3 minutes) and the supernatant was filtered with a 0.22 μm filter to obtain an HPLC sample. As a result of HPLC analysis, 0.74 mg of itraconazole was confirmed to be contained in 10 mg of the product. Yield: 7.4% (w/w).

<HPLC Analysis Conditions>
Instrument: Waters Alliance 2695 Separations Module, Waters 2487 Dual λ Absorbance Detector
Column: XBridge C18, particle size: 3.5 μm, 4.6 mm×100 mm (Waters)
Mobile phase: A: 0.2% diisopropylamine-methanol solution, B: 0.5% aqueous ammonium acetate solution, A:B=4:1 (v/v)
Flow rate: 0.9 ml/min
Detection wavelength: 263 nm
Retention time: 3.0 min

### (3) Measurement of Particle Diameter Distribution

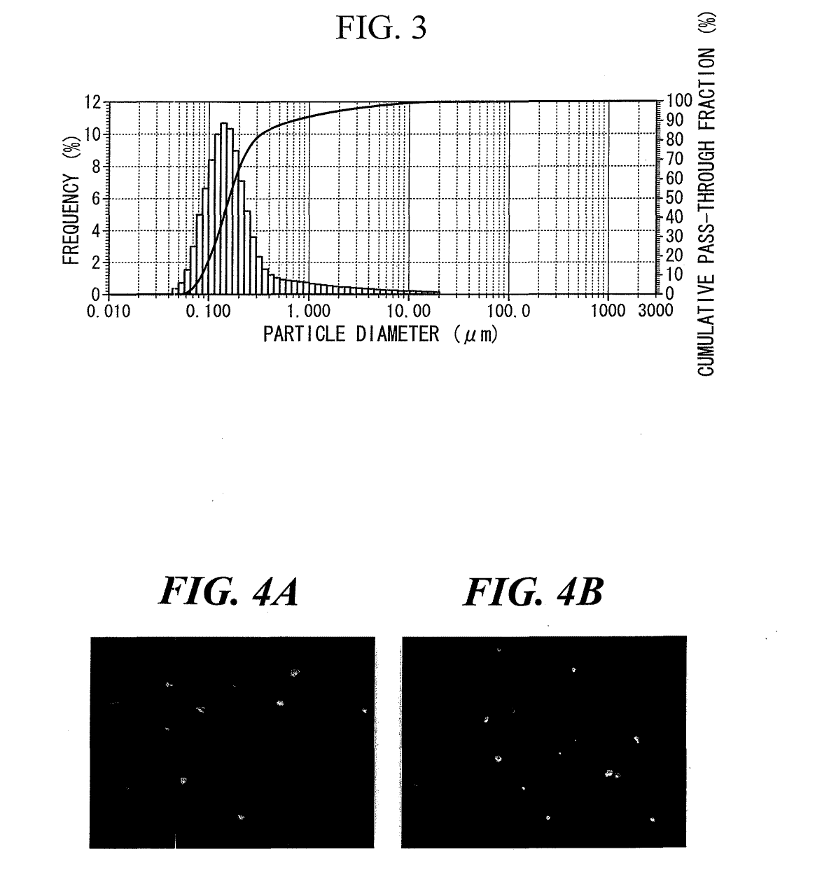

1 mg of the product was suspended in Milli-Q water (15 ml) and irradiated for 5 minutes with ultrasonic waves (frequency: 28 kHz, output: 100 W) followed by measurement of particle diameter. The results are shown in FIG. 3.

### Example 4

### Preparation of Substance Composed of PEG-Modified HAP and siRNA

(1) Preparation of Substance Composed of PEG-Modified HAP and Fluorescently Labeled siRNA

6 mg of PEG-modified HAP were weighed out followed by the addition of 10 ml of pure water. The mixture was transferred to an emulsifier-disperser and treated for 1 minute at 16000 rpm to obtain a homogeneous suspension. 18 μl of an aqueous solution of 10 mg/ml fluorescently labeled siRNA were added to 3 ml of the suspension and mixed well.

### (2) Fluorescence Microscope Observation

6 ml of glycerin were added to 3 ml of the product. The sample was observed with a fluorescence microscope. The fluorescence excitation wavelength was set to 490 nm for a fluorescein-labeled sample and 550 nm for a rhodamine-labeled sample. The results are shown in FIG. 4.

### (3) Results

Observation by fluorescence microscopy revealed the siRNA to be coated onto the surface of the PEG-modified HAP.

### Example 5

### Blood Kinetics Study of Substance Composed of PEG-Modified HAP and Itraconazole During Administration to Rats

### (1) Purpose

Changes in blood concentrations of a substance composed of PEG-modified HAP and itraconazole were confirmed by intravenous and oral administration to rats followed by calculation of bioavailability.

### (2) Test Substances

(1) Intravenous Administration

Aqueous suspension of substance composed of PEG-modified HAP and itraconazole

Storage conditions: Blocked from light, room temperature

(2) Oral Administration

Aqueous suspension of substance composed of PEG-modified HAP and itraconazole

Storage conditions: Blocked from light, room temperature

### (3) Animals

Species: Rat, strain: SD, sex: males
No. of animals: n=3
Age at dosing: 7 weeks
Feeding conditions at dosing: Non-fasting
Dosage:
Intravenous administration: 5 mg/2 ml/kg (intravenous injection using 27G injection needle)
Oral administration: 12 mg/4.8 ml/kg

### (4) Blood Collection

Plasma collection times: 0.5, 2, 6, 18, 24, 48 and 168 hours after dosing

Plasma collection: Approx. 0.5 ml of blood were drawn from a caudal vein using a capillary tube treated with sodium heparin

Plasma obtained by centrifuging the blood (12000 rpm, 4° C., 3 minutes) was stored frozen at −20° C. until the time of measurement.

(5) Observation of Symptoms: The animals were only observed for general condition, and observation of specific sites or specific tissues was not carried out.

### (6) Measurement of Plasma Concentration

Analysis Method:
Measured substance: Itraconazole
Standard substance: Itraconazole
Storage conditions: Cool, dark location
Internal standard: Loratadine
Storage conditions: Cool, dark location
Analysis conditions: LC/MS/MS

### (7) LC/MS/MS Conditions

Column: Capcell Pak C18 MG II, 50 mm×4.6 mm, i.d.: 5 mm (Shiseido)
Mobile phase: A: 2 mmol/L ammonium acetate, B: acetonitrile A:B=35:65 (v/v)
Flow rate: 0.5 ml/min
Ion source: APCI
Polarity: Positive ion
Detected ions: m/z 705.1, 392.1 (itraconazole)
   m/z 383.5, 337.2 (internal standard, I.S.)

### (8) Pretreatment

100 μl of I.S. solution were added to a calibration curve sample and measurement sample and stirred. 100 μl of acetonitrile were added to a blank sample followed by stirring.
700 μl of acetonitrile were added and stirred.
The mixture was centrifuged for 10 minutes at about 12,000 g (4° C.)
5 μl of supernatant were injected into the LC/MS/MS.

### (9) Results

The substance composed of PEG-modified HAP and itraconazole was observed to demonstrate effects that improve intestinal absorption, demonstrating high bioavailability of about 57% at 0.5 to 24 hours (Table 1).

Based on the results of an in vivo blood kinetics study on commercially available Itrizole for injection and Itrizole 50 capsules conducted simultaneous to the above study, the bioavailability at 0.5 to 48 hours of oral Itrizole 50 capsules versus Itrizole for injection was about 39%.

On the basis of these results, a substance composed of submicron-sized PEG-modified HAP and a poorly soluble pharmaceutical was indicated to be useful as a novel injection preparation and as a superior oral preparation exhibiting high oral absorption.

Table 1

### Example 6

### Blood Kinetics Study of Substance Composed of PEG-Modified HAP and Clarithromycin During Administration to Rats

### (1) Purpose

Changes in blood concentrations of a substance composed of PEG-modified HAP and clarithromycin were confirmed by intravenous and subcutaneous administration to rats.

### (2) Test Substances

(1) Intravenous Administration

Aqueous suspension of substance composed of PEG-modified HAP and clarithromycin

Storage conditions: Blocked from light, room temperature

(2) Subcutaneous Administration

Aqueous suspension of substance composed of PEG-modified HAP and clarithromycin

Storage conditions: Blocked from light, room temperature

### (3) Animals

Species: Rat, strain: SD, sex: males
No. of animals: n=3
Age at dosing: 7 weeks
Feeding conditions at dosing: Non-fasting
Dosage:
Intravenous administration: 1 mg/ml/kg (intravenous injection using 27G injection needle)
Subcutaneous administration: 2 mg/2 ml/kg (subcutaneous injection using 27G injection needle)

### (4) Blood Collection

Plasma collection times: 0.5, 2, 6, 12, 24, 48 and 168 hours after dosing

Plasma collection: Approx. 0.5 ml of blood were drawn from a caudal vein using a Pasteur pipette treated with sodium heparin

Plasma obtained by centrifuging the blood (8000×g, 4° C., 3 minutes) was stored frozen at −20° C. until the time of measurement.

(5) Observation of Symptoms: The animals were only observed for general condition, and observation of specific sites or specific tissues was not carried out.

### (6) Measurement of Plasma Concentration

Analysis Method:
Measured substance: Clarithromycin
Standard substance: Clarithromycin
Storage conditions: Cool, dark location
Internal standard: Erythromycin B Storage conditions: Cool, dark location
Analysis conditions: LC/MS/MS

### (7) LC/MS/MS Conditions

Column: Atlantis dC18 3 mm, 4.6 mm i.d.×75 mm (Waters)
Guard column: Atlantis dC18 3 mm, 4.6 mm i.d.×20 mm (Waters)
Mobile phase: A: 20 mmol/L ammonium formate, B: acetonitrile
A:B=55:45 (v/v)
Flow rate: 0.5 ml/min
Ion source: ESI
Polarity: Positive ion
Detected ions: m/z 748.6, 158.2 (clarithromycin)
   m/z 718.6, 158.3 (Internal standard, I.S.)

### (8) Pretreatment

1. 200 μl of 5% (w/v) sodium carbonate and 4 μl of ethyl acetate were added to a calibration curve sample, blank sample and measurement sample.
2. The mixture was shaken for about 15 minutes at room temperature followed by centrifuging for 10 minutes at room temperature and about 1800×g.
3. The supernatant (organic layer) was transferred to a 13 ml polypropylene (p.p.) tube.
4. The supernatant was concentrated and dried to a solid in flowing nitrogen (40° C., approx. 30 minutes).
5. 1 ml of reconstitution solution were added to the residue and stirred.
6.10 μl were injected into the LC/MS/MS.

### (9) Results

As shown in Table 2, the substance composed of submicron-sized PEG-modified HAP and a poorly soluble pharmaceutical in the form of clarithromycin was indicated to be useful as a novel injection preparation (able to be intravenously or subcutaneously injected using a 27G injection needle).

Table 2

### Example 7

### Preparation of Substance Composed of PEG-Modified HAP and Candesartan

### (1) Preparation of Composition

A composition was prepared using a method similar to Example 2 or Example 3.

### (2) Measurement of Drug Adsorption Rate

Drug adsorption rate was confirmed using a method similar to Example 2 or Example 3. Adsorption rate: 7.4% (w/w)

### Example 8

### Preparation of Substance Composed of PEG-Modified HAP and Candesartan Cilexetil

### (1) Preparation of Composition

A composition was prepared using a method similar to Example 2 or Example 3.

### (2) Measurement of Drug Adsorption Rate

Drug adsorption rate was confirmed using a method similar to Example 2 or Example 3. Adsorption rate: 6.3% (w/w)

### Example 9

### Preparation of Substance Composed of PEG-Modified HAP and

| | |
|---|---|
| 5′-GUGAAGUCAACAUGCCUGCTT-3′ | (SEQ ID NO. 1) |
| and | |
| 5′-GCAGGCAUGUUGACUUCACTT-3′ | (SEQ ID NO. 2)] |

### (1) Preparation of Composition

A composition was prepared using a method similar to Example 4.

### (2) Measurement of Drug Adsorption Rate

Drug adsorption rate was confirmed by fluorescence analysis. Adsorption rate: 20% (w/W)

### Example 10

### Preparation of Substance Composed of PEG-Modified HAP and

| | |
|---|---|
| 5′-CUUACGCUGAGUACUUCGATT-3′ | (SEQ ID NO. 3) |
| and | |
| 5′-UCGAAGUACUCAGCGUAAGTT-3′ | (SEQ ID NO. 4)] |

### (1) Preparation of Composition

A composition was prepared using a method similar to Example 4.

### (2) Measurement of Drug Adsorption Rate

Drug adsorption rate was confirmed by fluorescence analysis. Adsorption rate: 20% (w/w)

### Example 11

### Preparation of Substance Composed of PEG-Modified HAP and Etoposide

### (1) Preparation of Composition

A composition was prepared using a method similar to Example 2 or Example 3.

### (2) Measurement of Drug Adsorption Rate

Drug adsorption rate was confirmed using a method similar to Example 2 or Example 3. Adsorption rate: 8.0% (w/w)

### Example 12

### Preparation of Substance Composed of PEG-Modified HAP and Nelfinavir Mesylate

### (1) Preparation of Composition

A composition was prepared using a method similar to Example 2 or Example 3.

### (2) Measurement of Drug Adsorption Rate

Drug adsorption rate was confirmed using a method similar to Example 2 or Example 3. Adsorption rate: 7.4% (w/w)

### Example 13

### Preparation of Substance Composed of PEG-Modified HAP and Simvastatin

### (1) Preparation of Composition

A composition was prepared using a method similar to Example 2 or Example 3.

### (2) Measurement of Drug Adsorption Rate

Drug adsorption rate was confirmed using a method similar to Example 2 or Example 3. Adsorption rate: 7.9% (w/w)

### Example 14

### Preparation of Substance Composed of PEG-Modified HAP and 7-ethyl-10-hydroxy-Camptothecine

### (1) Preparation of Composition

A composition was prepared using a method similar to Example 2 or Example 3.

### (2) Measurement of Drug Adsorption Rate

Drug adsorption rate was confirmed using a method similar to Example 2 or Example 3. Adsorption rate: 6.8% (w/w)

### Example 15

### Preparation of Substance Composed of PEG-Modified HAP and Paclitaxel

### (1) Preparation of Composition

A composition was prepared using a method similar to Example 2 or Example 3.

### (2) Measurement of Drug Adsorption Rate

Drug adsorption rate was confirmed using a method similar to Example 2 or Example 3. Adsorption rate: 7.4% (w/w)

### Example 16

### Preparation of Substance Composed of PEG-Modified HAP and Saquinavir Mesylate

### (1) Preparation of Composition

A composition was prepared using a method similar to Example 2 or Example 3.

### (2) Measurement of Drug Adsorption Rate

Drug adsorption rate was confirmed using a method similar to Example 2 or Example 3. Adsorption rate: 8.0% (w/w)

### Example 17

### Preparation of Substance Composed of PEG-Modified HAP and Insulin

### (1) Preparation of Composition

A composition was prepared using a method similar to Example 4.

### (2) Measurement of Drug Adsorption Rate

Drug adsorption rate was confirmed using a method similar to Example 2 or Example 3. Adsorption rate: 12.7% (w/w)

### Example 18

### Elution Test of Substance Composed of PEG-Modified HAP And Candesartan Using the Paddle Method

(Results)

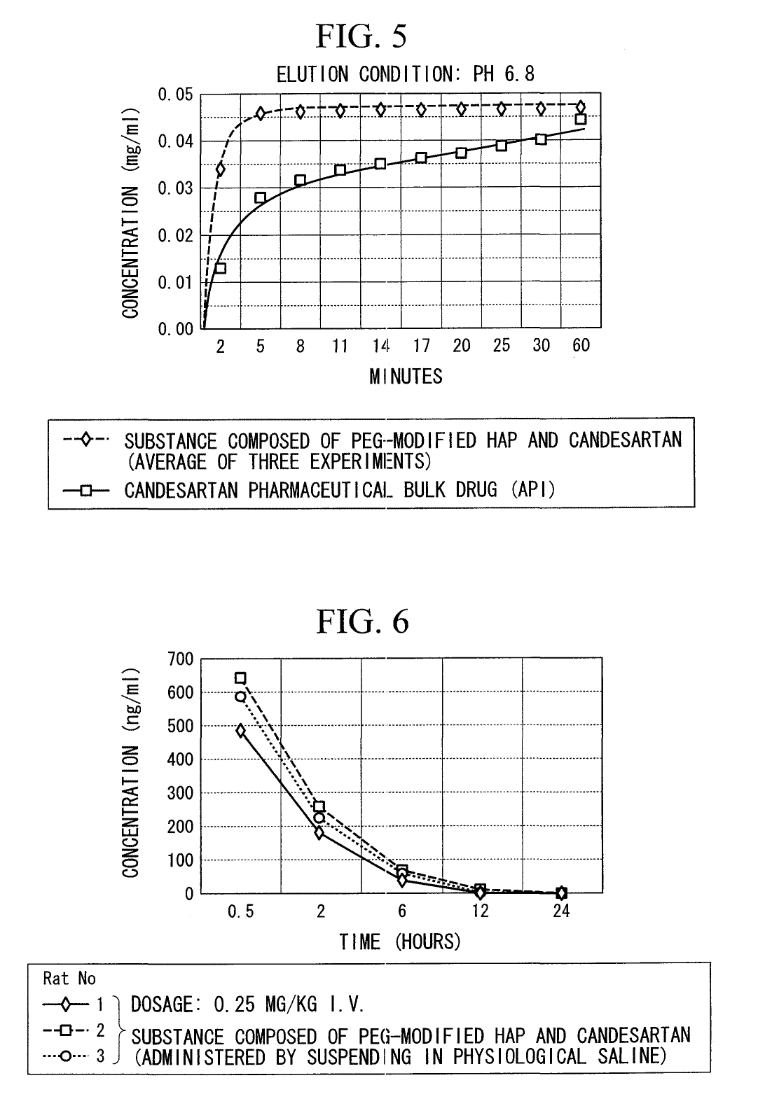

FIG. 5 is a graph showing eluted concentration of candesartan over time (min).

As shown in FIG. 5, in comparison to elution of candesartan from a candesartan pharmaceutical bulk drug requiring about 1 hour, candesartan rapidly eluted from a substance composed of PEG-modified HAP and candesartan, being completely eluted in about 5 minutes.

### Example 19

### Blood Kinetics Study of Substance Composed of PEG-Modified HAP and Candesartan During Intravenous (I.V.) Administration to Rats

(Results)

FIG. 6 is a graph showing time-based concentration changes (hours) in plasma following intravenous injection to rats.

As shown in FIG. 6, a substance composed of submicron-sized PEG-modified HAP and a poorly soluble pharmaceutical in the form of candesartan was indicated to be useful as a novel injection preparation (able to be intravenously or subcutaneously injected using a 27G injection needle).

### Example 20

### Blood Kinetics Study of Substance Composed of PEG-Modified HAP and Candesartan During Oral (P.O.) Administration to Rats

(Results)

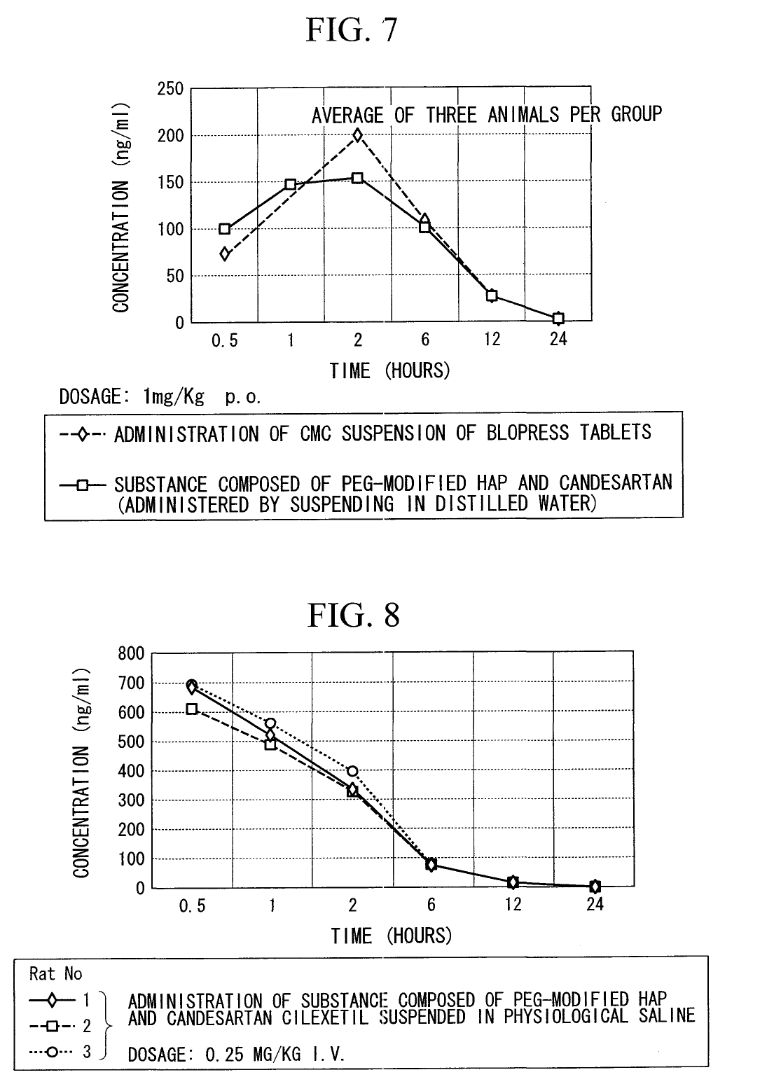

FIG. 7 is a graph showing time-based concentration changes (hours) in plasma following oral administration to rats.

As shown in FIG. 7, a substance composed of PEG-modified HAP and candesartan was indicated to demonstrate oral absorption comparable to commercially available Blopress (registered trademark) tablets.

### Example 21

### Blood Kinetics Study of Substance Composed of PEG-Modified HAP and Candesartan Cilexetil During I.V. Administration to Rats

(Results)

FIG. 8 is a graph showing time-based concentration changes (hours) in plasma following intravenous injection to rats.

As shown in FIG. 8, a substance composed of PEG-modified HAP and candesartan cilexetil was indicated to be useful as a novel injection preparation (able to be intravenously or subcutaneously injected using a 27G injection needle).

### Example 22

### Blood Kinetics Study of Substance Composed of PEG-Modified HAP and Candesartan Cilexetil During P.O. Administration to Rats

(Results)

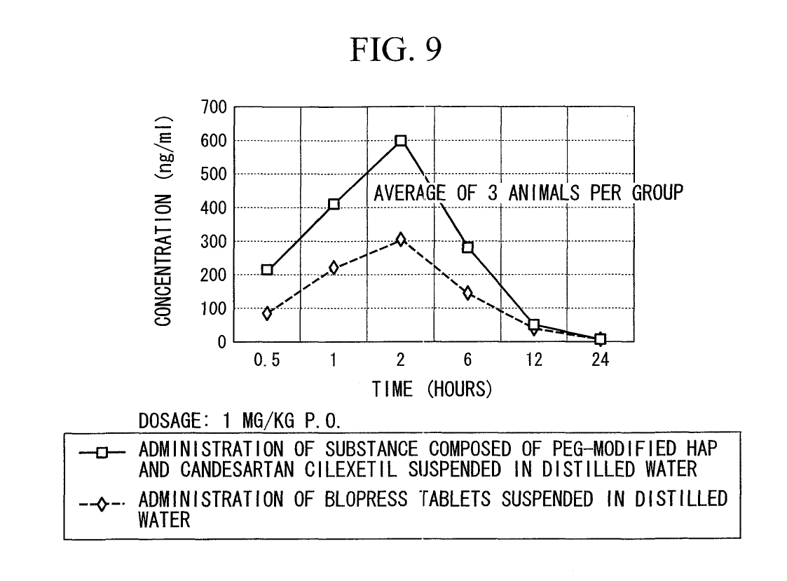

FIG. 9 is a graph showing time-based concentration changes (hours) in plasma following oral administration to rats.

As shown in FIG. 9, a substance composed of PEG-modified HAP and candesartan cilexetil demonstrated oral absorption roughly 1.5 times that of commercially available Blopress tablets.

### Example 23

### In Vitro Cytotoxicity Evaluation Study of Substance Composed of PEG-Modified HAP and

| | |
|---|---|
| 5′-GUGAAGUCAACAUGCCUGCTT-3′ | (SEQ ID NO. 1) |
| and | |
| 5′-GCAGGCAUGUUGACUUCACTT-3′ | (SEQ ID NO. 2)] |

(Results)

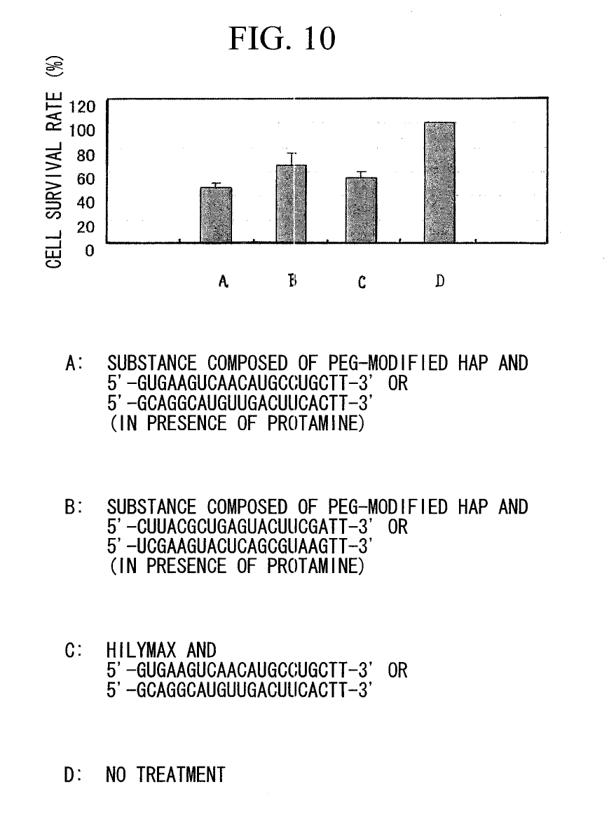

FIG. 10 is a graph showing cytotoxicity against A549 cells.

As shown in FIG. 10A, a substance composed of PEG-modified HAP and 5′-GUGAAGUCAACAUGCCUGCTT-3′ (SEQ ID NO. 1) and 5′-GCAGGCAUGUUGACUUCACTT-3′ (SEQ ID NO. 2) demonstrated a 50% cell survival rate against A549 human lung cancer cells, and indicated potent effects comparable to the positive control shown in FIG. 10C (HilyMax manufactured by Dojindo Laboratories). On the other hand, the negative control shown in FIG. 10B demonstrated a cell survival rate of about 70%.

### Example 24

### Transfection Test of Substance Composed of PEG-Modified HAP and Fluorescently Labeled

| | |
|---|---|
| 5′-CUUACGCUGAGUACUUCGATT-3′ | (SEQ ID NO. 3) |
| and | |
| 5′-UCGAAGUACUCAGCGUAAGTT-3′ | (SEQ ID NO. 4) |
| and | |

### Fluorescence Microscope Observation

(Results)

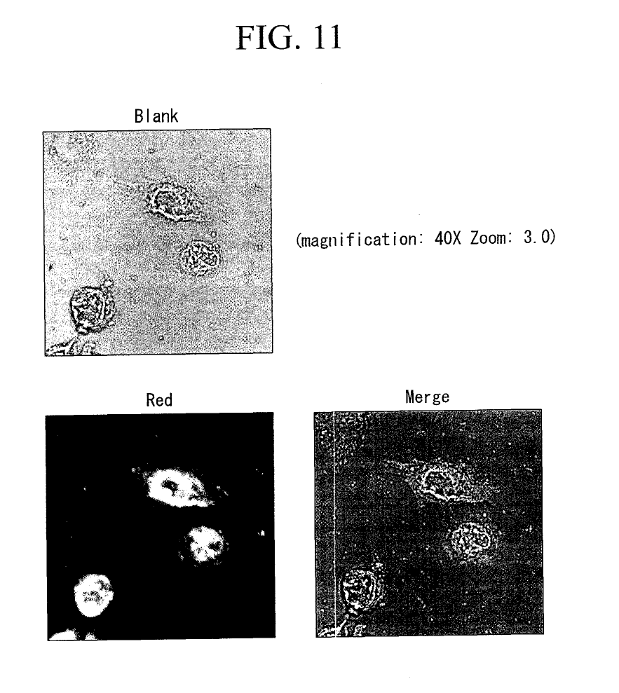

Fluorescence micrographs of cells 4 hours after addition of a substance composed of PEG-modified HAP and fluorescently labeled 5′-CUUACGCUGAGUACUUCGATT-3′ (SEQ ID NO. 3) and 5′-UCGAAGUACUCAGCGUAAGTT-3′ (SEQ ID NO. 4) to A549 human lung cancer cells are shown in FIG. 11.

### Example 25

### Blood Kinetics Study of Substance Composed of PEG-Modified HAP and Etoposide During I.V. Administration to Rats

(Results)

As shown in Table 3, a substance composed of submicron-sized PEG-modified HAP and a poorly soluble pharmaceutical in the form of etoposide was observed to demonstrate higher accumulation of etoposide in the liver as compared with a commercially available etoposide injection preparation (Vepesid injection).

Table 3

### Example 26

### Microscopic Observation of Substance Composed of PEG-Modified HAP and Simvastatin

(Results)

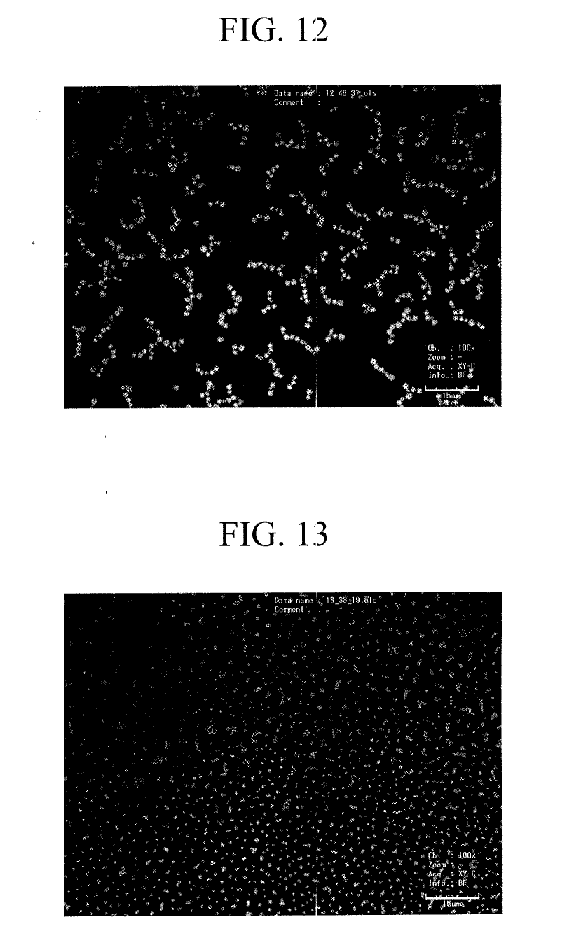

A confocal laser micrograph of a substance composed of PEG-modified HAP and simvastatin is shown in FIG. 12. Particles of the substance composed of submicron-sized PEG-modified HAP and simvastatin having a uniform particle diameter were observed to exhibit Brownian movement.

### Example 27

### Microscopic Observation of Substance Composed of PEG-Modified HAP and Nelfinavir Mesylate

(Results)

A confocal laser micrograph of a substance composed of PEG-modified HAP and nelfinavir mesylate is shown in FIG. 13. Particles of the substance composed of submicron-sized PEG-modified HAP and nelfinavir mesylate having a uniform particle diameter were observed to exhibit Brownian movement.

### Example 28

### Preparation of Substance Composed of PEG-Modified HAP and Bromocriptine Mesylate

### (1) Preparation of Composition

A composition was prepared using a method similar to Example 2 or Example 3.

### (2) Measurement of Drug Adsorption Rate

Drug adsorption rate was confirmed using a method similar to Example 2 or Example 3. Adsorption rate: 4.2% (w/w)

### INDUSTRIAL APPLICABILITY

Use of the PEG-modified HAP of the present invention as a base material enables even a poorly soluble pharmaceutical substance to be treated in the manner of a soluble substance, facilitating administration of a drug into the body and improving blood retention in the body.

1-23. (canceled)

24. A substance comprising:
hydroxyapatite having a particle diameter of 50 μm to 10 nm; and
a polyethylene glycol derivative having a carboxyl group as a terminal functional group, in which the carbon content is 0.1% to 10%.

25. A substance comprising hydroxyapatite and a polyethylene glycol derivative, said hydroxyapatite having a particle diameter of 50 μm to 10 nm, and being bonded to said polyethylene glycol derivative having a carboxyl group as a terminal functional group through —O(CO) bonds, wherein the carbon content is 0.1% to 10%.

26. A composition comprising:
the substance according to claim 24, and
a pharmaceutical active ingredient, and optionally a pharmaceutical additive, wherein the weight ratio of the pharmaceutical active ingredient is 1% to 30%.

27. A composition comprising:
the substance according to claim 25, and
a pharmaceutical active ingredient, and optionally a pharmaceutical additive, wherein the weight ratio of the pharmaceutical active ingredient is 1% to 30%.

28. The composition according to claim 26, wherein the pharmaceutical active ingredient is selected from the group consisting of an siRNA, aptamer, RNA, DNA, peptide, and protein.

29. The composition according to claim 27, wherein the pharmaceutical active ingredient is selected from the group consisting of an siRNA, aptamer, RNA, DNA, peptide, and protein.

30. The composition according to claim 26, wherein the pharmaceutical active ingredient is clarithromycin, or itraconazole.

31. The composition according to claim 27, wherein the pharmaceutical active ingredient is clarithromycin, or itraconazole.

32. A method for obtaining a submicron-sized substance comprising hydroxyapatite and a polyethelene glycol derivative, said method comprising:
treating submicron-sized hydroxyapatite and an active ester of a polyethylene glycol derivative having a carboxyl group as a terminal functional group in an anhydrous organic solvent.

33. A method for obtaining the Composition according to claim 26, comprising:
treating the substance, the pharmaceutical active ingredient, and optionally the pharmaceutical additive, in an organic solvent.

34. A method for obtaining the composition according to claim 27, comprising:
treating the substance, the pharmaceutical active ingredient, and optionally the pharmaceutical additive, in an organic solvent.

35. The composition according to claim 26, wherein the pharmaceutical active ingredient is selected from the group consisting of candesartan, candesartan cilexetil, etoposide, nelfinavir mesylate, simvastatin, 7-ethyl-10-hydroxy-camptothecine, paclitaxel, saquinavir mesylate, insulin, bromocriptine mesylate, and a double-stranded siRNA having the sequence of:

| | |
|---|---|
| 5′-GUGAAGUCAACAUGCCUGCTT-3′, | (SEQ ID NO: 1) |
| and | |
| 5′-GCAGGCAUGUUGACUUCACTT-3′; | (SEQ ID NO: 2) |
| or | |
| 5′-CUUACGCUGAGUACUUCGATT-3′, | (SEQ ID NO: 3) |
| and | |
| 5′-UCGAAGUACUCAGCGUAAGTT-3′. | (SEQ ID NO: 4) |

36. The composition according to claim 27, wherein the pharmaceutical active ingredient is selected from the group consisting, of candesartan, candesartan cilexetil, etoposide, nelfinavir mesylate, simvastatin, 7-ethyl-10-hydroxy-camptothecine, paclitaxel, saquinavir mesylate, insulin, bromocriptine mesylate, and a double-stranded siRNA having the sequence of:

| | |
|---|---|
| 5′-GUGAAGUCAACAUGCCUGCTT-3′, | (SEQ ID NO: 1) |
| 5′-GCAGGCAUGUUGACUUCACTT-3′, | (SEQ ID NO: 2) |
| or | |
| 5′-CUUACGCUGAGUACUUCGATT-3′, | (SEQ ID NO: 3) |
| 5′-UCGAAGUACUCAGCGUAAGTT-3′. | (SEQ ID NO: 4) |


SEQUENCE LISTING (4 sequences)

SEQ ID NO: 1 - DNA, 21 bp, Artificial Sequence
  Description of Combined DNA/RNA Molecule Synthetic oligonucleotide
gugaagucaacaugccugctt

SEQ ID NO: 2 - DNA, 21 bp, Artificial Sequence
  Description of Combined DNA/RNA Molecule Syntheticoligonucleotide
gcaggcauguugacuucactt

SEQ ID NO: 3 - DNA, 21 bp, Artificial Sequence
  Description of Combined DNA/RNA Molecule Syntheticoligonucleotide
cuuacgcugaguacuucgatt

SEQ ID NO: 4 - DNA, 21 bp, Artificial Sequence
  Description of Combined DNA/RNA Molecule Syntheticoligonucleotide
ucgaaguacucagcguaagtt